(19) United States
(12) Patent Application Publication
(10) Pub. No.: US 2020/0100657 A1
(43) Pub. Date: Apr. 2, 2020

(54) PROTECTION COVER FOR NOSE ENDOSCOPY

(71) Applicant: CHONBUK NATIONAL UNIVERSITY HOSPITAL, Jeonju-si (KR)

(72) Inventors: Sang Kyi LEE, Jeonju-si (KR); Myoung Hwan KO, Jeonju-si (KR); Kap Soo HAN, Suwon-si (KR)

(73) Assignee: CHONBUK NATIONAL UNIVERSITY HOSPITAL, Jeonju-si (KR)

(21) Appl. No.: 16/613,431

(22) PCT Filed: May 15, 2018
(86) PCT No.: PCT/KR2018/005560

§ 371 Date: Nov. 14, 2019

(30) Foreign Application Priority Data
May 24, 2017 (KR) 10-2017-0063940

(51) Int. Cl. A61B 1/00; A61B 1/233; A41D 13/11

(52) U.S. Cl. CPC A61B 1/00142; A41D 13/1169; A41D 13/1107; A61B 1/233

(57) ABSTRACT

A protection cover for a nose endoscopy, and more particularly to a protection cover for a nose endoscopy that can prevent discharges generated during sneezing in a nose endoscopy from being ejected to a healthcare provider. The protection cover for a nose endoscopy includes: a fixed member coupled to the head of a wearer; and a main cover coupled to a lower portion of the fixed member and configured to cover a front surface of the face of the wearer, A through-hole for exposing the nostrils of the wearer to the outside is formed in the main cover.

### CROSS-REFERENCES TO THE RELATED APPLICATIONS

This application is the national phase entry of International Application No. PCT/KR2018/005560, filed on May 15, 2018, which is based upon and claims priority to Korean Patent Application No. 10-2017-0063940, filed on May 24, 2017, the entire contents of which are incorporated herein by reference.

### TECHNICAL FIELD

The present invention relates to a protection cover for a nose endoscopy, and more particularly to a protection cover for a nose endoscopy that can prevent discharges generated during sneezing in a nose endoscopy from being ejected to a healthcare provider.

### BACKGROUND

Endoscopes currently used in the medical field are mechanisms that are inserted into the interior of an organ such that the interior of the organ can be observed by using an image through a display when it is difficult to observe the surgical part of a patient by naked eyes.

In recent years, diagnoses for directly observing an internal organ and performing a tissue test have become possible due to the development of electronic endoscopes, and have replaced surgeries due to the rapid development of the surgical endoscopes.

Further, nasal endoscopes are being currently widely used to identify nasal diseases.

When an endoscope is inserted into a nose for a test, a tip of the endoscope stimulates a nasal cavity so that the patient tends to sneeze.

Then, the discharges generated during sneezing of the patent may be directly injected to the healthcare provider.

### SUMMARY

The present invention has been made in an effort to solve the above-described problems, and provides a protection cover for a nose endoscopy that can facilitate the nose endoscopy and can prevent discharges from being injected to a healthcare provider when a patient sneezes as well.

A protection cover for a nose endoscopy may include: a fixed member coupled to the head of a wearer; and a main cover coupled to a lower portion of the fixed member and configured to cover a front surface of the face of the wearer, wherein a through-hole for exposing the nostrils of the wearer to the outside is formed in the main cover.

The main cover may include: a cover member of a transparent plastic coupled to a lower portion of the fixed member and configured to cover the front surface of the face; and a mask member coupled to the cover member and configured to cover a mouth portion of the wearer, and the through-hole for exposing the nostrils to the outside may be formed in the cover member, and the mask member is detachably coupled to the cover member.

The cover member may be detachably coupled to the fixed member.

A coupling part may be coupled to the fixed member to be rotatable upwards and downwards, and the cover member may be coupled to the coupling part.

A lower end of the cover member may be disposed above the mouth of the wearer, and the mask member may be detachably coupled to a lower portion of the cover member to cover the mouth portion of the wearer.

A lower end of the cover member may be disposed below the mouth of the wearer, and the mask member may be detachably coupled to an inner surface of the cover member to cover the mouth portion of the wearer.

The mask member may be formed of a material capable of absorbing a liquid.

The protection cover may further include: a protection member of an impact absorbing material coupled to an inside of the fixed member and configured to contact the forehead of the wearer, and the protection member may be detachably coupled to the fixed member.

The fixed member may include: a stripe member coupled to the head of the wearer; and a nose support coupled to the stripe member and supported by the bridge of the nose, and the stripe member may be disposed below the eyes of the wearer, and the main cover is coupled to a lower portion of the fixed member such that the upper end of the main cover is disposed below the eyes of the wearer and the lower end of the main cover is disposed below the mouth portion.

The main cover may be detachably coupled to the fixed member.

The main cover may include: a cover member of a plastic material coupled to a lower portion of the fixed member and configured to cover the front surface of the face, and in which the through-hole is formed; and a mask member detachably coupled to the inner surface of the cover member and configured to cover the mouth portion of the wearer.

The protection cover may further include: a nose covering member coupled to the fixed member or the protection cover and configured to cover the through-hole, and the nose covering member may be formed of a fiber material and covers the nose of the wearer.

The protection cover for a nose endoscopy of the present invention, which has been described above, has the following effects.

Because the cover member is formed of a transparent material and the cover member has the through-hole, a nose endoscope can be easily performed.

Further, when the patient sneezes in the process of a nose endoscopy, the discharges are attached to the mask member to be prevented from being ejected to the healthcare provider.

Further, because not the entire protection cover but only the part contaminated by the discharges is replaced, the purchase costs of the protection cover can be reduced.

### BRIEF DESCRIPTION OF THE DRAWINGS

The above and other objects, features, and advantages of the present invention will be more apparent from the following detailed description taken in conjunction with the accompanying drawings, in which:

FIG. 1 shows a perspective view of a protection cover for a nose endoscopy according to a first embodiment of the present invention;

FIG. 2 shows an exploded perspective view of the protection cover for a nose endoscopy according to the first embodiment of the present invention;

FIG. 3 shows a side view of a state in which the protection cover for a nose endoscopy is mounted according to the first embodiment of the present invention;

FIG. 4 shows a perspective view of a protection cover for a nose endoscopy according to a second embodiment of the present invention;

FIG. 5 shows an exploded perspective view of the protection cover for a nose endoscopy according to the second embodiment of the present invention;

FIG. 6 shows a side view of a state in which the protection cover for a nose endoscopy is mounted according to the second embodiment of the present invention;

FIG. 7 shows a perspective view of a protection cover for a nose endoscopy according to a third embodiment of the present invention; and

FIG. 8 shows a side view of a state in which the protection cover for a nose endoscopy is mounted according to the third embodiment of the present invention.

### DETAILED DESCRIPTION OF THE EMBODIMENTS

### First Embodiment

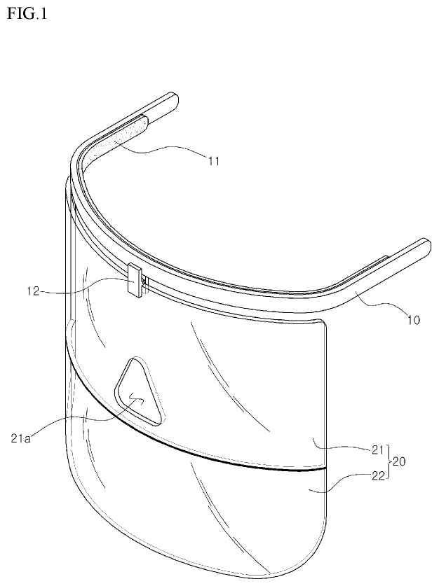

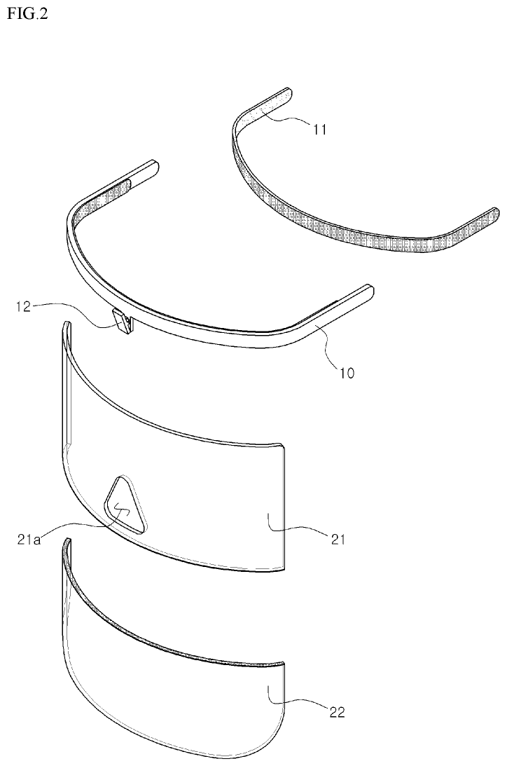

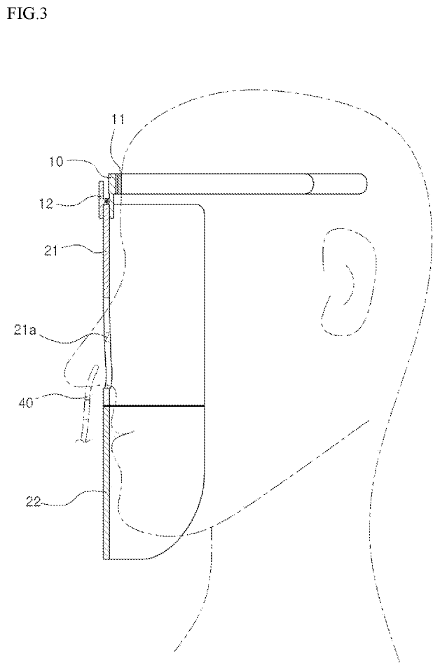

FIG. 1 is a perspective view of a protection cover for a nose endoscopy according to a first embodiment of the present invention. FIG. 2 is an exploded perspective view of the protection cover for a nose endoscopy according to the first embodiment of the present invention. FIG. 3 is a side view of a state in which the protection cover for a nose endoscopy is mounted according to the first embodiment of the present invention.

The protection cover for a nose endoscopy of the present invention includes a fixed member 10 and a main cover 20.

The fixed member 10 has an arc shape and is coupled to the head of a wearer by an elastic force or the like.

A protection member 11 of an impact absorbing material, which contacts the forehead of the wearer is coupled to an inside of the fixed member 10.

Then, the protection member 11 is detachably coupled to the fixed member 10 through a Velcro fastener or the like, and it is preferable that protection member 11 may be easily separated from the fixed member 10 or exchanged when the protection member 11 is contaminated or has to be replaced.

The main cover 20 is coupled to a lower portion of the fixed member 10 to cover the front surface of the face of the wearer, and includes a cover member 21 and a mask member 22.

The cover member 21 is coupled to a lower portion of the fixed member 10, and is formed of a transparent plastic material that covers the front surface of the face.

The cover member 21 is formed of a transparent material, and allows the wearer to secure the front field of view through the cover member 21 to prevent the wearer from fearing as the eyes of the wearer is blocked during a test.

A through-hole 21a for exposing the nostrils of the wearer to the outside is formed in the cover member 21.

Through the through-hole 21a, an endoscope 40 may be inserted into the nostril of the wearer from the outside.

Further, the cover member 21 may be fixedly coupled to the fixed member 10, and may be detachably coupled.

Because the cover member 21 is detachably coupled to the fixed member 10 through a Velcro fastener, a button, a zipper, or a snap button, only the cover member 21 may be easily separated from the fixed member 10 to be exchanged when the cover member 21 has to be replaced.

Further, because the cover member 21 is detachably coupled to the fixed member 10, the cover member 21 may be coupled to the fixed member 10 after the user wears the fixed member 10, which facilitates mounting.

Further, the cover member 21 may be simply detachably coupled to the fixed member 10, and as in the embodiment, the cover member 21 may be rotatably coupled to the fixed member 10.

In the embodiment, a coupling part 12 is rotatably coupled to the fixed member 10 upwards and downwards.

Further, the cover member 21 is coupled to the coupling part 12.

Because the cover member 21 is coupled to the fixed member 10 to be rotatable through the coupling part 12, the user may easily wear the fixed member 10 in the state in which the cover member 21 is rotated upwards.

An upper end of the coupling part 12 is hinge-coupled to the fixed member 10, and a lower end of the coupling part 12 has a shape such as a nipper such that the cover member 21 is detachably coupled.

The mask member 22 is coupled to the cover member 21 to cover a mouth portion of the wearer.

The mask member 22 is detachably coupled to the cover member 21.

In the embodiment, the lower end of the cover member 21 is disposed above the mouth of the wearer, and the mask member 22 is detachably coupled to a lower portion of the cover member 21 to cover the mouth portion of the wearer.

Because the mask member 22 that covers the mouth portion of the wearer is detachably coupled to a lower portion of the cover member 21, the discharges from the mouth when the wearer sneezes in the process of the nose endoscopy are attached to the mask member 22 not to be ejected to the healthcare providers.

Further, because the mask member 22 is detachably coupled to the cover member 21, only the mask member 22 may be separated from the cover member 21 to be replaced by a new one, which contributes to a sanitary aspect and decreases costs.

Further, it is preferable that the mask member 22 itself or the inner surface of the mask member 22 is formed of a material that may absorb a liquid, for example, cloth such that the discharges during the sneezing do not flow.

A gauze or the like may be coupled to an inner surface of the mask member 22.

Of course, the mask member 22 may be formed of plastic.

The mask member 22 is detachably coupled to the cover member 21 through various conventional known attachment/detachment structures such as a Velcro fastener, a button, a clip, or a hook structure.

Through the protection cover for a nose endoscopy of the present invention, the endoscope 40 may be easily inserted into the nostril through the through-hole 21a formed in the cover member 21, and the discharges may be attached to the mask member 22 to be prevented from being ejected to the healthcare providers when the patient sneezes in the process of the nose endoscope 40, which is sanitary.

Further, because the mask member 22, to which the discharges are attached, is separated from the cover member 21 to be replaced, only the contaminated parts may be replaced without having to replace the entire cover protection body.

In addition, because the cover member 21 is detachably coupled to or rotatably coupled to the fixed member 10, the wearer may easily wear the fixed member 10 without being interfered by the cover member 21.

### Second Embodiment

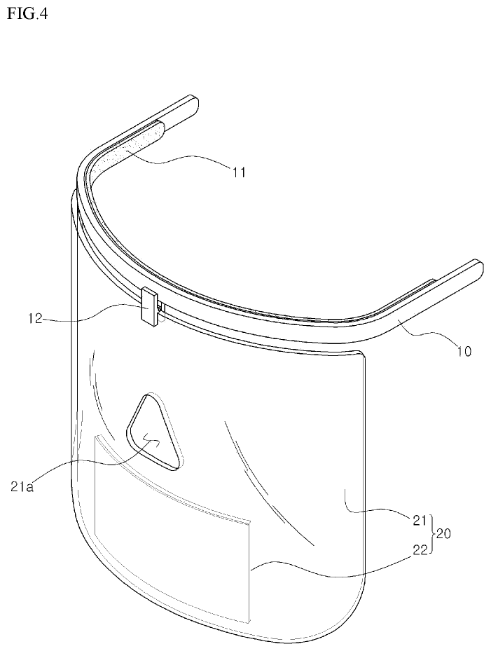

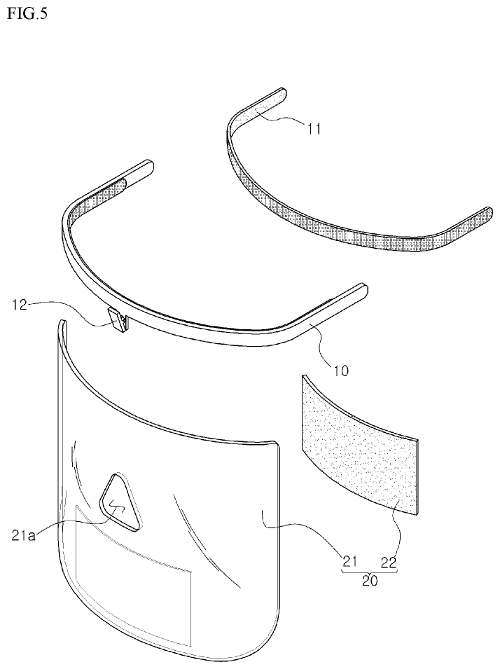

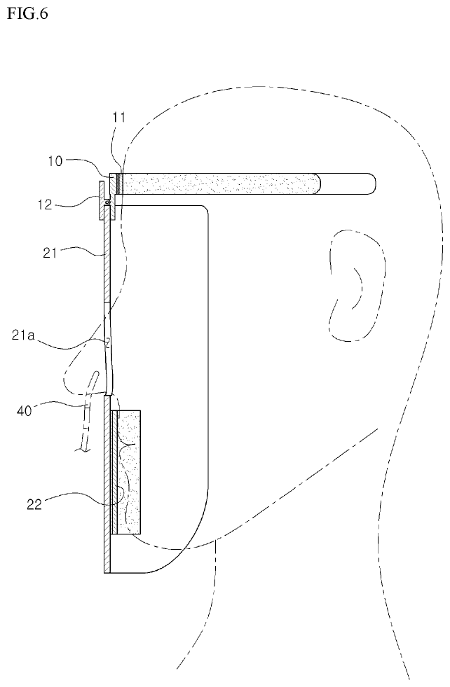

FIG. 4 is a perspective view of a protection cover for a nose endoscopy according to a second embodiment of the present invention. FIG. 5 is an exploded perspective view of the protection cover for a nose endoscopy according to the second embodiment of the present invention. FIG. 6 is a side view of a state in which the protection cover for a nose endoscopy is mounted according to the second embodiment of the present invention.

The second embodiment is different from the first embodiment in the main cover 20, that is, the cover member 21 and the mask member 22, and a description thereof will be mainly made.

A lower end of the cover member 21 is disposed below the mouth of the wearer.

That is, because the cover member 21 in the second embodiment is longer than the cover member 21 in the first embodiment, the mouth portion of the wearer is also covered by the cover member 21.

The mask member 22 is detachably coupled to the inner surface of the cover member 21 to be disposed at a mouth portion of the wearer.

Then, the mask member 22 is formed of a material that may absorb a liquid.

In this way, because the mask member 22 coupled to the inner surface of the cover member 21 is formed of a material that may absorb a liquid, for example, cloth, the discharges generated during sneezing are attached to be prevented from flowing.

In the embodiment, because the mask member 22 detachably coupled to the inner surface of the cover member 21 is separated to be replaced by a new one, it may be repeatedly and sanitarily used.

The other items are the same as or similar to the first embodiment, and a detailed description thereof will be omitted.

### Third Embodiment

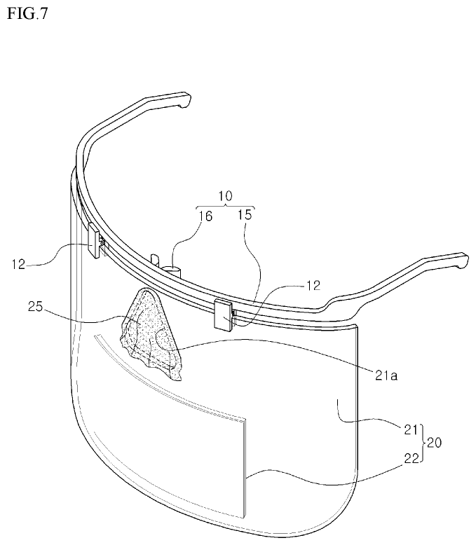

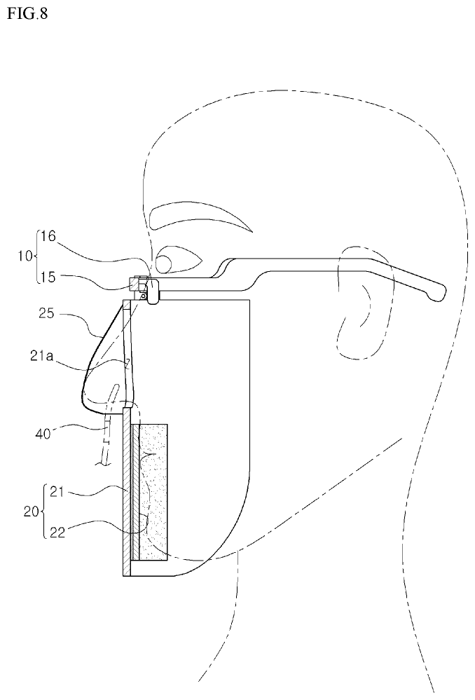

FIG. 7 is a perspective view of a protection cover for a nose endoscopy according to a third embodiment of the present invention. FIG. 8 is a side view of a state in which the protection cover for a nose endoscopy is mounted according to the third embodiment of the present invention.

The protection cover for a nose endoscopy according to the third embodiment of the present invention includes a fixed member 10 and a main cover 20.

The fixed member 10 includes a stripe member 15 and a nose support 16.

The stripe member 15 is coupled to the head of the wearer.

Preferably, the stripe member 15 is coupled to the ears of the wearer like the eyeglasses.

The nose support 16 is coupled to the stripe member 15 to contact the bridge of the nose of the wearer or be supported by the bridge of the nose.

The stripe member 15 is disposed below the eyes of the wearer on the front surface, and the field of view of the wearer is prevented from being hidden by the protection cover for a nose endoscopy of the present invention.

The main cover 20 is coupled to a lower portion of the fixed member 10, and the upper end of the main cover 20 is disposed below the eyes of the wearer and the lower end of the main cover 20 is disposed below the mouth portion of the wearer.

The main cover 20 is detachably coupled to the fixed member 10, in detail, the stripe member 15.

To achieve this, the coupling part 12, such as a nipper, is mounted to the stripe member 15, and the main cover 20 is detachably coupled to the stripe member 15 through the coupling part 12.

The main cover 20 includes a cover member 21 and a mask member 22.

The cover member 21 is formed of a plastic material, and is coupled to a lower portion of the fixed member 10 and covers the front surface of the face of the wearer.

A through-hole 21a for exposing the nose of the wearer to the outside is formed in the cover member 21.

The mask member 22 is detachably coupled to the inner surface of the cover member 21 to cover a mouth portion of the wearer.

The mask member 22 is formed of a material, to which spit discharged from the mouth of the wearer may be attached well.

Further, the nose covering member 25 that covers the through-hole 21a is coupled to the fixed member 10 or the protection cover.

In more detail, the nose covering member 25 is coupled to the stripe member 15 or the cover member 21.

The nose covering member 25 covers the through-hole 21a such that the discharges generated from the nose of the wearer during sneezing are attached to the nose covering member 25.

The nose covering member 25 is formed of a fabric material such as cloth, and naturally covers the nose of the wearer, which is exposed to the outside of the through-hole 21a.

The other items are the same as or similar to the first or second embodiment, and a detailed description thereof will be omitted.

The protection cover for a nose endoscopy of the present invention is not limited to the above-described embodiments, and may be variously modified within a range that is allowed by the technical sprit of the present invention.

### INDUSTRIAL APPLICABILITY

The present invention may be applied to a protection cover that is mounted during a nose endoscopy, and thus can be industrially applicable.

What is claimed is:

1. A protection cover for a nose endoscopy comprising:
a fixed member configured to be coupled to a head of a wearer; and
a main cover coupled to a lower portion of the fixed member and configured to cover a front surface of a face of the wearer,
wherein a through-hole for exposing a plurality of nostrils of the wearer to an outside is formed in the main cover.

2. The protection cover of claim 1, wherein the main cover comprises:
a cover member of a transparent plastic coupled to the lower portion of the fixed member and configured to cover the front surface of the face; and
a mask member coupled to the cover member and configured to cover a mouth portion of the wearer, and
wherein the through-hole for exposing the plurality of nostrils to the outside is formed in the cover member, and the mask member is detachably coupled to the cover member.

3. The protection cover of claim 2, wherein the cover member is detachably coupled to the fixed member.

4. The protection cover of claim 2, wherein a coupling part is coupled to the fixed member to be rotatable upwards and downwards, and the cover member is coupled to the coupling part.

5. The protection cover of claim 2, wherein a lower end of the cover member is configured to be disposed above the mouth portion of the wearer, and the mask member is detachably coupled to a lower portion of the cover member to cover the mouth portion of the wearer.

6. The protection cover of claim 2, wherein the lower end of the cover member is configured to be disposed below the mouth portion of the wearer, and the mask member is detachably coupled to an inner surface of the cover member to cover the mouth portion of the wearer.

7. The protection cover of claim 6, wherein the mask member is formed of a material capable of absorbing a liquid.

8. The protection cover of claim 2, further comprising:
a protection member of an impact absorbing material coupled to an inside of the fixed member and configured to contact a forehead of the wearer,
wherein the protection member is detachably coupled to the fixed member.

9. The protection cover of claim 1, wherein the fixed member comprises:
a stripe member configured to be coupled to the head of the wearer; and
a nose support coupled to the stripe member and configured to be supported by a bridge of the nose, and
wherein the stripe member is configured to be disposed below a plurality of eyes of the wearer, and the main cover is coupled to the lower portion of the fixed member such that an upper end of the main cover is configured to be disposed below the plurality of eyes of the wearer and a lower end of the main cover is configured to be disposed below the mouth portion.

10. The protection cover of claim 9, wherein the main cover is detachably coupled to the fixed member.

11. The protection cover of claim 9, wherein the main cover comprises:
a cover member of a plastic material coupled to the lower portion of the fixed member and configured to cover the front surface of the face; and
a mask member detachably coupled to the inner surface of the cover member and configured to cover the mouth portion of the wearer.

12. The protection cover of claim 9, further comprising:
a nose covering member coupled to the fixed member or the protection cover and configured to cover the through-hole,
wherein the nose covering member is formed of a fiber material and is configured to cover the nose of the wearer.
